(19) United States
(12) Patent Application Publication
(10) Pub. No.: US 2013/0295617 A1
(43) Pub. Date: Nov. 7, 2013

(54) METHODS AND COMPOSITIONS FOR PRODUCTION OF TREHALULOSE

(71) Applicant: Lauren Shaw, Durham, NC (US)

(72) Inventor: Lauren Shaw, Durham, NC (US)

(73) Assignee: SYNGENTA PARTICIPATIONS AG, Basel (CH)

(21) Appl. No.: 13/978,888

(22) PCT Filed: Mar. 5, 2012
(86) PCT No.: PCT/US12/27653

§ 371 Date: Jul. 10, 2013

Related U.S. Application Data
(60) Provisional application No. 61/450,193, filed on Mar. 8, 2011.

(51) Int. Cl. C12P 19/24

(52) U.S. Cl. CPC C12P 19/24
USPC 435/94

(57) ABSTRACT

The present invention provides a method of producing trehalulose, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose.

### STATEMENT OF PRIORITY

This application claims the benefit, under 35 U.S.C. §119 (e), of U.S. Provisional Application No. 61/450,193; filed Mar. 8, 2011, the entire contents of which is incorporated by reference herein.

### FIELD OF THE INVENTION

The present invention relates generally to methods and compositions for the production of trehalulose.

### BACKGROUND OF THE INVENTION

Trehalulose, or 1-O-α-D-glucopyranosyl-D-fructose, is a structural isomer of sucrose and exhibits properties that are advantageous as compared to sucrose. For example, trehalulose is acariogenic (causes less tooth decay), is digested and absorbed more slowly than sucrose and can attenuate insulin levels in the blood stream. Thus, trehalulose can be used as a substitute for sucrose in food and drinks for weight loss, sports and diabetics. Sucrose isomerase from bacterial sources is commonly used for production of trehalulose. In addition to trehalulose, sucrose isomerase enzymes can also produce isomaltulose (palatinose, 6-O-α-D-glucopyranosyl-D-fructose) and small amounts of glucose and fructose (Lee et al., Appl. Environ. Microbiol. 74:5183-5194 (2008)). The ratios of the reaction products of sucrose isomerase can vary according to which sucrose isomerase is used (i.e., the source of the sucrose isomerase) and the reaction conditions that are provided (Id.). The production of co-products does not reduce the efficiency of the production of trehalulose but the presence of these co-products in the trehalulose compositions can reduce the commercial value of the trehalulose. Thus, there is a need in the industry for methods that can provide trehalulose as the predominant product of sucrose isomerase.

Accordingly, the present invention addresses previous shortcomings in the art by providing methods for producing trehalulose as a predominant product of sucrose isomerase.

### SUMMARY OF THE INVENTION

The present invention provides a method of producing trehalulose, the method comprising, consisting essentially of, consisting of: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose.

Also provided herein is a method of producing trehalulose, the method comprising, consisting essentially of, consisting of: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the amount of sucrose in the sucrose-enzyme mixture is depleted by at least 75% as compared to the amount of sucrose in the sucrose solution of (a).

Additionally provided herein is a method for producing trehalulose, comprising, consisting essentially of, consisting of: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the amount of sucrose in the sucrose-enzyme mixture is depleted by at least 75% as compared to the amount of sucrose in the sucrose solution of (a) and further wherein the method also produces isomaltulose and the ratio of trehalulose to isomaltulose is about 95:5.

These and other aspects of the invention will be set forth in more detail in the description of the invention that follows.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Various embodiments of the invention are described herein. Any of the features of the various embodiments of the invention described herein can be combined, creating additional embodiments which are intended to be within the scope of the invention.

The present invention is based on the surprising discovery that providing non-optimal incubation conditions for sucrose isomerase enzymes (e.g., low temperatures) can provide an increased ratio of trehalulose to isomaltulose from sucrose. Thus, for example, under optimal conditions of pH 6 and 30° C., sucrose isomerase EcSI produces 60% trehalulose and 40% isomaltulose. In a further example, under optimal conditions of pH 6 and 40° C., the sucrose isomerase MX-45 produces 75-80% trehalulose to 9-11% isomaltulose. This compares to the production by both of these enzymes of at least about 95% trehalulose using the methods of the present invention. Further advantages of the methods of the present invention include that the sucrose is completely converted to product, the reaction is not product (feedback) inhibited, and the products remain stable (Le., are not further broken down into, for example, glucose, fructose).

Thus, in one aspect of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose.

In other embodiments, the sucrose-enzyme mixture is incubated at a temperature of about 0.5° C. to less than 10° C. Thus, in some embodiments, the sucrose-enzyme mixture is incubated at a temperature of about 0.5° C. to about 2° C., 0.5° C. to about 2.5° C., 0.5° C. to about 3° C., about 0.5° C. to about 4° C., about 0.5° C. to about 5° C., about 0.5° C. to about 6° C., about 0.5° C. to about 7° C., about 0.5° C. to about 8° C., about 0.5° C. to about 9° C., about 1° C. to about 3° C., about 1° C. to about 4° C., about 1° C. to about 5° C., about 1° C. to about 6° C., about 1° C. to about 7° C., about 1° C. to about 8° C., about 1° C. to about 9° C., about 2° C. to about 3° C., about 2° C. to about 4° C., about 2° C. to about 5° C., about 2° C. to about 6° C., about 2° C. to about 7° C., about 2° C. to about 8° C., about 2° C. to about 9° C., about 3° C. to about 4° C., about 3° C. to about 5° C., about 3° C. to about 6° C., about 3° C. to about 7° C., about 3° C. to about 8° C., about 3° C. to about 9° C., about 4° C. to about 5° C., about 4° C. to about 6° C., about 4° C. to about 7° C., about 4° C. to about 8° C., about 4° C. to about 9° C., about 5° C. to about 6° C., about 5° C. to about 7° C., about 5° C. to about 8° C., about 5° C. to about 9° C., about 6° C. to about 7° C., about 6° C. to about 8° C., about 6° C. to about 9° C., about 7° C. to about 8° C., about 7° C. to about 9° C., about 8° C. to about 9° C., and the like.

Thus, in other embodiments of this invention, the sucrose-enzyme mixture can be incubated at a temperature of about 0.5° C., about 1° C., about 1.5° C., about 2° C., about 2.5° C., about 3° C., about 3.5° C., about 4° C., about 4.5° C., about 5° C., about 5.5° C., about 6° C., about 6.5° C., about 7° C., about 7.5° C., about 8° C., about 8.5° C., about 9° C., about 9.5° C., or less than 10° C.

Accordingly, in some embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 0.5° C. to less than 10° C. for a period of time sufficient to convert the sucrose in the sucrose-enzyme mixture to a product comprising trehalulose. In other embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 0.5° C. to about 7° C. for a period of time sufficient to convert the sucrose in the sucrose-enzyme mixture to a product comprising trehalulose. In further embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 0.5° C. to about 5° C. for a period of time sufficient to convert the sucrose in the sucrose-enzyme mixture to a product comprising trehalulose. In additional embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 1° C. to about 5° C. for a period of time sufficient to convert the sucrose in the sucrose-enzyme mixture to a product comprising trehalulose.

In other embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature of about 1° C. for a period of time sufficient to convert the sucrose in the sucrose-enzyme mixture to a product comprising trehalulose. In other embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature of about 5° C. for a period of time sufficient to convert the sucrose in the sucrose-enzyme mixture to a product comprising trehalulose.

As discussed above, the optimal temperatures for sucrose isomerase enzymes are significantly higher than the temperatures used in the incubating step (b) of the present invention (e.g., 30° C.-40° C. as compared to 0.5° C.-10° C.). However, surprisingly, providing these reduced incubation temperatures results in an increased ratio of trehalulose to isomaltulose as compared to that produced under the optimal temperatures for the enzymes.

As used herein, the term “sucrose solution” refers to an aqueous medium (e.g., water, buffered water) in which sucrose is dissolved so as to obtain a sucrose solution of a particular concentration of sucrose. In some embodiments of this invention, the sucrose solution of (a) (i.e., the sucrose solution which is contacted with a sucrose isomerase enzyme) comprises sucrose at an initial concentration of about 0.01M to a saturated solution. Thus, in some embodiments of this invention, the sucrose solution of (a) can comprise sucrose at an initial concentration in the range of about 0.01M to about 0.1M, about 0.01M to about 0.2M, about 0.01M to about 0.3M, about 0.01M to about 0.4M, about 0.01M to about 0.5M, about 0.01M to about 0.6M, about 0.01M to about 0.7M, about 0.01M to about 0.8M, about 0.01M to about 0.9M, about 0.01M to about 1M, about 0.01M to about 2M, about 0.01M to about 3M, about 0.01M to about 4M, about 0.01M to about 5M, about 0.01M to about 6M, about 0.01M to about 7M, about 0.01M to about 8M, about 0.01M to about 9M, about 0.01M to about 10M, about 0.01M to about 10M, about 0.1M to about 0.3M, about 0.1M to about 0.4M, about 0.1M to about 0.4M, about 0.1M to about 0.5M, about 0.1M to about 0.6M, about 0.1M to about 0.7M, about 0.1M to about 0.8M, about 0.1M to about 0.9M, about 0.1M to about 1M, about 0.1M to about 2M, about 0.1M to about 3M, about 0.1M to about 4M, about 0.1M to about 5M, about 0.1M to about 6M, about 0.1M to about 7M, about 0.1M to about 8M, about 0.1M to about 9M, about 0.1M to about 10M, about 0.1M to about 10M, and the like.

Therefore, in some embodiments of this invention, the initial sucrose concentration in the sucrose solution of (a) can be about 0.01M, about 0.02M, about 0.03M, about 0.04M, about 0.05M, about 0.06M, about 0.07M, about 0.08M, about 0.09M, about 0.1M, about 0.15M, about 0.2M, about 0.25M, about 0.3M, about 0.35M, about 0.4M, about 0.45M, about 0.5M, about 0.55M, about 0.6M, about 0.65M, about 0.7M, about 0.75M, about 0.8M, about 0.85M, about 0.9M, about 0.95M, about 1M, about 1.1M, about 1.2M, about 1.3M, about 1.4M, about 1.5M, about 1.6M, about 1.7M, about 1.8M, about 1.9M, about 2M, about 2.1M, about 2.2M, about 2.3M, about 2.4M, about 2.5M, about 2.6M, about 2.7M, about 2.8M, about 2.9M, about 3M, about 3M, about 3.1M, about 3.2M, about 3.3M, about 3.4M, about 3.5M, about 3.6M, about 3.7M, about 3.8M, about 3.9M, about 4M, about 4.1M, about 4.2M, about 4.3M, about 4.4M, about 4.5M, about 4.6M, about 4.7M, about 4.8M, about 4.9M, about 5M, about 5.1M, about 5.2M, about 5.3M, about 5.4M, about 5.5M, about 5.6M, about 5.7M, about 5.8M, about 5.9M, about 6M, about 6.1M, about 6.2M, about 6.3M, about 6.4M, about 6.5M, about 6.6M, about 6.7M, about 6.8M, about 6.9M, about 7M, about 7.1M, about 7.2M, about 7.3M, about 7.4M, about 7,5M, about 7.6M, about 7.7M, about 7.8M, about 7.9M, about 8M, about 8.1M, about 8.2M, about 8.3M, about 8.4M, about 8.5M, about 8.6M, about 8.7M, about 8.8M, about 8.9M, about 9M, about 9.1M, about 9.2M, about 9.3M, about 9.4M, about 9.5M, about 9.6M, about 9.7M, about 9.8M, about 9.9M, about 10.0M, and the like.

As described above, in some embodiments, the sucrose solution can be saturated. As used herein, “a saturated solution” is a solution in which the maximum amount of sucrose is dissolved in a given quantity of solvent (e.g., water, buffer, and the like) at a given temperature. Thus, in some embodiments the initial sucrose concentration in the sucrose solution of (a) is the concentration that results when a maximum amount of sucrose is dissolved in a solvent (e.g., water, buffer, and the like) at a given temperature. Thus, for example, at 1° C. a saturated sucrose solution has a concentration of about 1.9 M.

In some embodiments of this invention, the pH of incubating step (b) is about 5 to about 9. Thus, in some embodiments of this invention, the pH of the incubating step (b) is about 5, about 5.5, about 6, about 6.5, about 7, about 7.5, about 8, about 8.5, or about 9. In other embodiments, the pH of the incubating step of (b) is about 6 to about 7. In still other embodiments, the pH of the incubating step of (b) is about 6.

In additional embodiments of this invention, the sucrose isomerase enzyme in the sucrose-enzyme mixture of step (a) has a range of activity of about 0.01 units/mg of enzyme (protein) to about 1000 units/mg of protein. As used herein, one unit is defined as an amount of enzyme required to liberate 1 μmol of substrate (e.g., sucrose) per minute at an assay temperature.

Thus, the sucrose isomerase enzyme of the present invention can be provided in an amount from about 0.01 units to about 1000 units, about 0.1 units to about 750 units, about 1 unit to about 600 units, or about 5 units to about 50 units of enzyme activity per mole substrate. Thus, in some embodiments, a suitable enzyme dosing can be about 0.01 units to 1000 units per μmol sucrose. In other embodiments, a suitable enzyme dose is 0.1 units to 750 units per μmol of sucrose. Alternatively, in further embodiments of the invention, the compositions can include about 0.01 unit to about 1 unit, about 1 unit to about 10 units, about 10 units to about 100 units, about 100 units to about 200 units, about 200 units to about 300 units, about 300 units to about 400 units, about 400 units to about 500 units, about 500 units to about 600 units, about 600 units to about 700 units, about 700 units to about 800 units, about 800 units to about 900 units, about 900 units to about 1000 units, or more of enzyme activity per μmol of sucrose. In some embodiments of this invention, a suitable enzyme dose is 600 units per μmol of sucrose.

The activity of a sucrose isomerase enzyme can be determined using HPAEC (high performance anion exchange chromatography) on a Dionex ICS3000 system. HPAEC with a CarboPac PA20 column is used to detect the trehalulose product. Trehalulose can be eluted from the column with a 40 mM NaOH, 4 mM NaOAc isocratic gradient

If the activity of the enzyme composition is insufficient, it can be concentrated prior to use. Methods of concentrating polypeptides and proteins such as enzymes are well known in the art. See, e.g., Ahmed, Principles and Reactions of Protein Extraction, Purification, and Characterization (CRC Press 2004); Dennison, A Guide to Protein Isolation, 2^{nd} ed. (Kluwer Academic Publishers 2003); Degerli & Akpinar (2001) Anal. Biochem. 297:192-194; and Protein Methods, 2^{nd} ed. (Bollag et al. eds., Wiley Publishers 1996). For example, ultrafiltration can be used to concentrate a supernatant of the enzyme 5-25 fold.

In another aspect, the present invention provides a method of producing trehalulose, wherein the amount of sucrose in the sucrose-enzyme mixture is depleted by at least 75% as compared to the amount of sucrose in the sucrose solution in step (a) that is contacted with the sucrose isomerase enzyme. In other aspects of this invention, the sucrose in the sucrose-enzyme mixture is depleted by an amount of about 75% to about 100%. The % sucrose (measured in mM) is the sucrose present in the solution after the reaction has taken place as compared to the initial sucrose concentration (mM). Thus, in further embodiments, the sucrose-enzyme mixture is depleted by about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, and the like. As described herein, these embodiments can be combined with any other embodiments, such as for example, any of the temperatures or temperature ranges of the incubation step, to create additional embodiments.

Thus, for example, in some embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the amount of sucrose in the sucrose-enzyme mixture is depleted by at least 75% as compared to the amount of sucrose in the sucrose solution of (a). In other embodiments of this invention, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 0.5° C. to about 5° C. for a period of time sufficient to convert the sucrose in the sucrose-enzyme mixture to a product comprising trehalulose, wherein the amount of sucrose in the sucrose-enzyme mixture is depleted by at least 75% as compared to the amount of sucrose in the sucrose solution of (a).

In additional embodiments, a method of producing trehalulose is provided, the method comprising: (a) contacting a sucrose solution with a sucrose isomerase enzyme to produce a sucrose-enzyme mixture; and (b) incubating the sucrose-enzyme mixture of (a) at a temperature in a range of about 0.5° C. to about 5° C. for a period of time sufficient to convert the sucrose in the sucrose-enzyme mixture to a product comprising trehalulose, wherein the amount of sucrose in the sucrose-enzyme mixture is depleted by 75% to about 100% as compared to the amount of sucrose in the sucrose solution of (a).

As used herein, the term “depleted” means reduced or decreased in amount when comparing a second time point to a first time point in an assay. Thus, for example, the amount of sucrose in the sucrose enzyme solution of part (b) can be depleted or reduced in amount at a given time point as compared to the amount of sucrose initially provided in the sucrose solution of part (a).

In another aspect of this invention, the period of time sufficient to deplete the amount of sucrose in the sucrose-enzyme mixture is the time period necessary to deplete the sucrose in the sucrose-enzyme mixture by an amount by at least 75%, from about 75% to about 100%, from about 95% to about 100%, and the like. Thus, in some embodiments, the period of time sufficient to deplete the amount of sucrose in the sucrose-enzyme mixture is the time period necessary to deplete the sucrose in the sucrose-enzyme mixture by an amount of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range therein, and the like. In some embodiments of this invention, the period of time sufficient to deplete the amount of sucrose in the sucrose-enzyme mixture is the time period necessary to deplete the sucrose in the sucrose-enzyme mixture by an amount of about 95% to about 100% (e.g., about 95%, about 96%, about 97%, about 98%, about 99%, about 100%. In other embodiments, the period of time sufficient to deplete the amount of sucrose in the sucrose-enzyme mixture is the time period necessary to deplete the sucrose in the sucrose-enzyme mixture by an amount of about 100%.

Accordingly, the period of time sufficient to reduce the amount of sucrose in the sucrose-enzyme mixture as described above can be in a range from about 24 hours to about 96 hours.

Thus, for example, the period of time sufficient to reduce the amount of sucrose in the sucrose-enzyme mixture can be about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, 53 about hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 85 hours, about 90 hours, about 95 hours, about 100 hours, or any range therein, and the like. In some aspects of this invention, the period of time sufficient to reduce the amount of sucrose in the sucrose-enzyme mixture can be about 48 hours to about 96 hours. In further aspects of this invention, the period of time sufficient to reduce the amount of sucrose in the sucrose-enzyme mixture can be about 60 hours to about 72 hours. In other aspects of this invention, the period of time sufficient to reduce the amount of sucrose in the sucrose-enzyme mixture can be at least 70 hours.

The sucrose isomerase enzyme of the present invention can be any sucrose isomerase from any organism. Sucrose isomerase is also known as isomaltulose synthase or a-glucosyltransferase (EC.5.4.99.11) (See also, the enzyme database available at www.enzyme-database.org/). In some embodiments of this invention, the sucrose isomerase can be from Bemisia argentifoli, Erwinia rhapontici, Erwinia caratovera, Erwinia caratovera EcS1, Azotobacter vinelandii, Protoaminobacter rubrum, Pantoea dispersa, Serratia plymuthica, Klebsiella planticola, Klebsiella sp., Klebsiella terrigena, Pseudomonas mesoacidophila, Agrobacterium radiobacter, Pseudomonas mesoacidophila, Pseudomonas mesoacidophila (MX-45), Pseudomonas putida, Pimelobacter spp., Thermus sp. ATCC 43814, Thermus sp. ATCC 43815, Thermus aquaticus, Thermus filiformis, Thermus rubis, or any combination thereof. Methods for growing organisms such as those that produce sucrose isomerase enzymes are well known in the art.

Accordingly, some non-limiting examples of a sucrose isomerase of this invention include a sucrose isomerase from Raoultella planticola, Genbank Accession No: AAP57085.1, GI: 37908601; asucrose isomerase from Erwinia rhapontici, Genbank Accession No:AAP57084.1, GI: 37903482; a sucrose isomerase from Pantoea dispersa, Genbank Accession No: AAP57083.1; GI: 37903467; a sucrose isomerase from Leucaena leucocephala, Genbank Accession No: ABB01680.1, GI: 77737727; a sucrose isomerase from Erwinia rhapontici, Genbank Accession No: ADJ56407.2, GI: 323903042; a sucrose isomerase from Pectobacterium atrosepticum SCRI1043, Genbank Accession No: YP_{—}049947.1, GI: 50120780; a sucrose isomerase from Enterobacter sp. FMB-1, Genbank Accession No: ACF42098.1, GI: 194307174; a sucrose isomerase from Pectobacterium atrosepticum SCRI1043, Genbank Accession No: CAG74753.1; GI: 49611306; a sucrose isomerase from Erwinia rhapontici, Genbank Accession No: AAK28735.1, GI: 13517315; a sucrose isomerase from Azotobacter vinelandii DJ, Genbank Accession No: ACO77078.1, GI: 226717907; a sucrose isomerase from Erwinia sp. Ejp617, Genbank Accession No: ADP12651.1, GI: 310767701; a sucrose isomerase from Erwinia amylovora ATCC 49946, Genbank Accession No: YP_{—}003538902.1; GI: 292899533; a sucrose isomerase from Azotobacter vinelandii DJ, Genbank Accession No: YP_{—}002798053.1, GI: 226942980; a sucrose isomerase Erwinia tasmaniensis Et1/99, Genbank Accession No: YP_{—}001907593.1, GI: 188533796; a sucrose isomerase from Erwinia pyrifoliae Ep1/96, Genbank Accession No: YP_{—}002648731.1; GI: 259908375; a sucrose isomerase from Erwinia pyrifoliae Ep1/96, Genbank Accession No: CAX55502.1, GI: 224963997; a sucrose isomerase from Erwinia amylovora ATCC 49946, Genbank Accession No: CBJ46498.1, GI: 291199381; a sucrose isomerase from Pectobacterium carotovorum subsp. brasiliensis PBR1692, Genbank Accession No: ZP_{—}03825380,1; GI: 227111724; a sucrose isomerase from Erwinia tasmaniensis Et1/99, Genbank Accession No: CA096700.1; GI: 188028838; and the like, or any combination thereof.

This invention is further envisioned to encompass the use of one or more sucrose isomerase enzymes, wherein the one or more sucrose isomerase enzymes can be obtained from the same or different organisms. Thus, any combination of different or the same sources can be used for the enzymes of the present invention. In some embodiments, the one or more sucrose isomerase enzymes can be produced by and obtained from a single organism. In other embodiments, wherein more than one sucrose isomerase enzyme is used, the sucrose isomerase enzymes can be produced by different organisms. In still other embodiments, wherein more than one sucrose isomerase enzyme is used, the sucrose isomerase enzymes can be produced by the same type of organism (e.g., bacteria) but which are from different families or genera. In further embodiments, wherein more than one sucrose isomerase enzyme is used, the sucrose isomerase enzymes can be produced by different species within the same genus or can be produced by the same species but different strains or varieties of that species.

Thus, non-limiting examples of combinations of sucrose isomerase enzymes from different sources include a sucrose isomerase enzyme from an insect in combination with a sucrose enzyme from a bacterium, a sucrose isomerase enzyme from Erwinia spp. in combination with a sucrose enzyme from a Pseudomonas spp., a sucrose isomerase enzyme from an Erwinia spp. in combination with both a sucrose enzyme from Pseudomonas spp. and a sucrose isomerase enzyme from Protoaminobacter spp., a sucrose isomerase enzyme from Erwinia caratovera in combination with a sucrose enzyme from Erwinia rhapontici, and the like.

The sucrose isomerase enzymes can be isolated from an organism and provided in the composition of the present invention as a partially or fully purified full-length enzyme, or as an active variant or fragment thereof, or the enzyme can be provided as a cell extract or cell lysate from the cell or cells of an organism that produces sucrose isomerase enzyme(s). In some embodiments, the sucrose isomerase can be produced by an organism transformed with a nucleotide encoding a sucrose isomerase enzyme of the invention. Complete purification is not required in any case. The enzyme, variant or fragment therefore can be at least about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% pure (w/w), or more.

A “cell extract” or “cell lysate” as used herein, refers to an extract or lysates from one or more of the cells of an organism that produces at least one sucrose isomerase enzyme useful with the present invention. Thus, for example, a cell extract/lysate can refer to an extract/lysate derived from one or more disrupted cells of a microorganism or other organism that produces a sucrose isomerase. Methods for disrupting cells are well known in the art and include, but are not limited to, osmotic shock, bead method (i.e., “beadbeating”), sonication, detergent, French press, freeze-thaw, enzymatic treatment, and/or high shear mechanical methods. The cell extract/lysate can be used as is or can be purified further using techniques known in the art for protein purification and as described herein.

Thus, in some embodiments, the present invention provides a method for producing trehalulose, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the sucrose isomerase enzyme is from Bemisia argentifoli, Erwinia rhapontici, Erwinia caratovera, Erwinia caratovera EcS1, Azotobacter vinelandii, Protoaminobacter rubrum, Pantoea dispersa, Serratia plymuthica, Klebsiella planticola, Klebsiella sp., Klebsiella terrigena, Pseudomonas mesoacidophila, Agrobacterium radiobacter, Pseudomonas mesoacidophila, Pseudomonas mesoacidophila (MX-45), Pseudomonas putida, Pimelobacter spp., Thermus sp. ATCC 43814, Thermus sp. ATCC 43815, Thermus aquaticus, Thermus filiformis, Thermus rubis, or any combination thereof.

In further embodiments of this invention, a method is provided for producing trehalulose, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the amount of sucrose in the sucrose-enzyme mixture is depleted by at least 75% as compared to the amount of sucrose in the sucrose solution of (a) and the sucrose isomerase enzyme is from Bemisia argentifoli, Erwinia rhapontici, Erwinia caratovera, Erwinia caratovera EcS1, Azotobacter vinelandii, Protoaminobacter rubrum, Pantoea dispersa, Serratia plymuthica, Klebsiella planticola, Klebsiella sp., Klebsiella terrigena, Pseudomonas mesoacidophila, Agrobacterium radiobacter, Pseudomonas mesoacidophila, Pseudomonas mesoacidophila (MX-45), Pseudomonas putida, Pimelobacter spp., Thermus sp. ATCC 43814, Thermus sp. ATCC 43815, Thermus aquaticus, Thermus filiformis, Thermus rubis, or any combination thereof. Methods for growing organisms such as those that produce sucrose isomerase enzymes are well known in the art.

Methods of purifying polypeptides and proteins such as enzymes are well known in the art. See, e.g., Cesar & Mr\check{s}a (1996) Enzyme Microb. Tech. 19:289-296; Chivero et al. (2001) Food Chem. 72:179-185; de Albuquerque Lucena-Neto et al. (2004) Br. J. Microb. 35:86-90; Ehle & Horn (1990) Bioseparation 1:97-110; Gupta et al. (2002) Biotechnol. Lett. 24:2005-2009; Hengen (1995) Trends Biochem Sci. 20:285-286; Kanda et al. (1985) J. Biochem. 98:1545-1554; Khasin et al. (1993) Appl. Environ. Microb. 59:1725-1730; Kudo et al. (1985) J. Gen. Microbiol. 131:2825-2830; Regnier (1983) Science 222:245-252; Roy & Uddin (2004) Pak. J. Biol. Sci. 7:372-379; Royer & Nakas (1991) Eu. J. Biochem. 202:521-529; Sá-Pereira et al. (2003) Mol. Biotechnol. 24:257-281; Shaw, “Peptide purification by reverse-phase HPLC” 257-287 In: Methods in Molecular Biology, Vol. 32 (Walker ed., Humana Press 1994); Simpson et al. (1991) Biochem. J. 277:419-417; Encyclopedia of Chemical Technology, 52^{nd} ed. (Kirk-Othmer ed., Wiley-Interscience 2007); Basic Methods in Protein Purification and Analysis: A Laboratory Manual (Simpson et al. eds., Cold Spring Harbor Laboratory Press 2008); Enzyme Purification and Related Techniques. Methods in Enzymology, Vol. 22 (Jakoby ed., Academic Press Inc. 1971) Methods in Enzymology: Affinity Techniques—Enzyme Purification: Part B: Vol. 34: Affinity Techniques Part B (Kaplan et al. eds., Elsevier 1974); as well as US Patent Application Publication Nos. 2009/0137022 and 2009/0239262; and U.S. Pat. Nos. 4,347,322; 4,634,673; 4,725,544 and 5,437,992.

Examples of purification techniques suitable for enzymes include, but are not limited to, precipitation such as ammonium sulfate precipitation, separation based on molecular size such as gel filtration, separation based on charge such as ion-exchange chromatography, separation based on specific interaction with other biomolecules such as bio-affinity chromatography or antibody recognition of amino acid sequence, separation based on other principles such as hydrophobic interaction chromatography or hydroxyapatite chromatography and separation based on electrophoretic principles (e.g., acrylamide, starch electrophoresis).

In other embodiments, the present invention provides a method for producing trehalulose, wherein the method also produces isomaltulose. In some embodiments of this invention, wherein both trehalulose and isomaltulose are produced, the ratio of trehalulose to isomaltulose is about 95:5. In other embodiments of this invention, wherein both trehalulose and isomaltulose are produced, the ratio of trehalulose to isomaltulose is about 96:4, about 97:3, about 98:2, about 99:1 and the like. In some aspects of this invention, the method for producing trehalulose also produces about 1% total of glucose and fructose.

Accordingly, in some embodiments, the present invention provides a method for producing trehalulose, the method comprising: (a) contacting a sucrose solution with a sucrose isomerase enzyme to produce a sucrose-enzyme mixture; and (b) incubating the sucrose-enzyme mixture of (a) at a temperature of less than 10° C. for a period of time sufficient to convert the sucrose in the sucrose-enzyme mixture to a product comprising trehalulose, wherein the method also produces isomaltulose. In some embodiments of this invention, wherein both trehalulose and isomaltulose are produced, the ratio of trehalulose to isomaltulose is about 95:5.

### Definitions:

As used herein, “about” means within a statistically meaningful range of a value such as a stated concentration, time frame, weight (e.g., a percentage change (reduction or increase in weight)), volume, temperature or pH. Such a range can be within an order of magnitude, typically within 20%, more typically still within 10%, and even more typically within 5% of a given value or range. The allowable variation encompassed by “about” will depend upon the particular system under study, and can be readily appreciated by one of skill in the art.

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim “and those that do not materially affect the basic and novel characteristic(s)” of the claimed invention. Thus, the term “consisting essentially of” when used in a claim of this invention is not intended to be interpreted to be equivalent to “comprising.”

The terms “polypeptide,” “protein,” and “peptide” refer to a chain of covalently linked amino acids. Unless otherwise indicated, the term “polypeptide” encompasses both peptides and proteins. In general, the term “peptide” can refer to shorter chains of amino acids (e.g., 2-50 amino acids); however, all three terms overlap with respect to the length of the amino acid chain. Polypeptides, proteins, and peptides may comprise naturally occurring amino acids, non-naturally occurring amino acids, or a combination of both. The polypeptides, proteins, and peptides may be isolated from sources (e.g., cells or tissues) in which they naturally occur, produced recombinantly in cells in vivo or in vitro or in a test tube in vitro, or synthesized chemically. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

The term “isolated” as used herein can further refer to a nucleic acid molecule, nucleotide sequence, polypeptide, peptide or fragment thereof that is substantially free of cellular material, viral material, and/or culture medium, or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose.

Accordingly, “isolated” refers to a nucleic acid molecule, nucleotide sequence, polypeptide, peptide or fragment that is altered “by the hand of man” from the natural state; i.e., that, if it occurs in nature, it has been changed or removed from its original environment, or both. For example, a naturally occurring polynucleotide or a polypeptide naturally present in a living organism in its natural state is not “isolated”, but the same polynucleotide or polypeptide separated from the coexisting materials of its natural state is “isolated”, as the term is employed herein. For example, with respect to polynucleotides, the term isolated means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

A variant of an enzyme of this invention is biologically active and therefore possesses the desired activity of the reference enzyme (e.g., sucrose isomerase activity) as described herein. The variant can result from, for example, a genetic polymorphism or human manipulation. A biologically active variant of the reference enzyme can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference enzyme as determined by sequence alignment programs and parameters described elsewhere herein. An active variant can differ from the reference enzyme sequence by as few as 1-15 amino acid residues, as few as 1-10, such as 6-10, as few as 5, as few as 4, 3, 2, or even 1 amino acid residue.

Naturally occurring variants may exist within a population. Such variants can be identified by using well-known molecular biology techniques, such as the polymerase chain reaction (PCR), and hybridization as described below. Synthetically derived nucleotide sequences, for example, sequences generated by site-directed mutagenesis or PCR-mediated mutagenesis which still encode a sucrose isomerase, are also included as variants. One or more nucleotide or amino acid substitutions, additions, or deletions can be introduced into a nucleotide or amino acid sequence disclosed herein, such that the substitutions, additions, or deletions are introduced into the encoded protein. The additions (insertions) or deletions (truncations) may be made at the N-terminal or C-terminal end of the native protein, or at one or more sites in the native protein. Similarly, a substitution of one or more nucleotides or amino acids may be made at one or more sites in the native protein.

For example, conservative amino acid substitutions may be made at one or more predicted, preferably nonessential amino acid residues. A “nonessential” amino acid residue is a residue that can be altered from the wild-type sequence of a protein without altering the biological activity, whereas an “essential” amino acid is required for biological activity. A “conservative amino acid substitution” is one in which the amino acid residue is replaced with an amino acid residue with a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

For example, amino acid sequence variants of the reference enzyme can be prepared by mutating the nucleotide sequence encoding the enzyme. The resulting mutants can be expressed recombinantly, and screened for those that retain biological activity by assaying for xylanase and cellulase activity using standard assay techniques. Methods for mutagenesis and nucleotide sequence alterations are known in the art. See, e.g., Kunkel (1985) Proc. Natl. Acad. Sci. USA 82:488-492; Kunkel et al. (1987) Methods in Enzymol. 154:367-382; and Techniques in Molecular Biology (Walker & Gaastra eds., MacMillan Publishing Co. 1983) and the references cited therein; as well as U.S. Pat. No. 4,873,192. Clearly, the mutations made in the DNA encoding the variant must not disrupt the reading frame and preferably will not create complimentary regions that could produce secondary mRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (National Biomedical Research Foundation, Washington, D.C.), herein incorporated by reference.

The deletions, insertions and substitutions of the enzymes described herein are not expected to produce radical changes in the characteristics of the enzymes (e.g., the enzyme activity). However, when it is difficult to predict the exact effect of the substitution, deletion or insertion in advance of doing so, one of skill in the art will appreciate that the effect can be evaluated by routine screening assays that can screen for the particular enzyme activities of interest (e.g., sucrose isomerase activity).

As noted above, the compositions can comprise active fragments of the sucrose isomerase enzyme. As used herein, “fragment” means a portion of the reference enzyme that retains sucrose isomerase activity. A fragment also means a portion of a nucleic acid molecule encoding the reference enzyme. An active fragment of the enzyme can be prepared, for example, by isolating a portion of an enzyme-encoding nucleic acid molecule that expresses the encoded fragment of the enzyme (e.g., by recombinant expression in vitro), and assessing the activity of the fragment. Nucleic acid molecules encoding such fragments can be at least about 150, 200, 250, 300, 350, 400, 450, 500, 550, 600, 650, 700, 800, 900, 1,000, 1,100, 1,200, 1,300 or 1,400 contiguous nucleotides, or up to the number of nucleotides present in a full-length enzyme-encoding nucleic acid molecule. As such, polypeptide fragments can be at least about 50, 60, 70, 80, 90, 100, 125, 150, 175, 200, 225 or 250 contiguous amino acid residues, or up to the total number of amino acid residues present in the full-length enzyme.

The present invention is more particularly described in the following examples that are intended as illustrative only since numerous modifications and variations therein will be apparent to those skilled in the art.

### EXAMPLES

### Example 1

### Small Scale Reaction Volume (100 uL) with EcSI Enzyme

Microbially expressed and purified Erwinia caratovora sucrose isomerase (EcSI) activity was tested across a temperature range of 30° C. to 1° C. The assay was carried out in a volume of 100 μl and comprised 584 mM sucrose, 6 ug/mL pure EcSI in 0.1 M phosphate/citrate buffer, pH 6. The assay was carried out for 70 hr in a thermocycler.

TABLE 1
Trehalulose production as
a function of reaction temperature.

| Temperature | % TRE | % ISM |
|---|---|---|
| 30 deg | 62.43 | 37.57 |
| 25 deg | 71.10 | 28.90 |
| 20 deg | 79.00 | 21.00 |
| 15 deg | 83.92 | 16.08 |
| 10 deg | 89.39 | 10.61 |
| 8 deg | 91.83 | 8.17 |
| 6 deg | 92.73 | 7.27 |
| 4 deg | 93.53 | 6.47 |
| 1 deg | 94.82 | 4.99 |

Ninety-five percent pure trehalulose was produced by the ESCI sucrose isomerase by carrying out the sucrose isomerase reaction at 1° C. until the sucrose substrate was 100% depleted (depletion was complete by about 48 hours).

### Example 2

### Sucrose Isomerase Reaction Using Sucrose Isomerase MX-45

The EcSI enzyme was overexpressed in the pET24 bacterial expression system and the enzyme was purified via affinity chromatography against a C-terminal His-tag.

Microbially expressed MX-45 cell lysate activity was tested across a temperature range of 40° C. to 1° C. The assay was carried out in a volume of 10 ml and comprised 584 mM sucrose and a 10% cell lysate in 0.1 M phosphate/citrate buffer, pH 6. The assay was performed in a chiller filled with ethylene glycol and water for 70 hr. The results are provided in Table 2.

TABLE 2
Percent trehalulose
produced using sucrose isomerase
MX-45 and varying temperatures.

| Temperature | % trehalulose |
|---|---|
| 40 deg | 81 |
| 28 deg | 85 |
| 1 deg | 95 |

Ninety-five percent pure trehalulose was produced by the MX-45 sucrose isomerase by carrying out the sucrose isomerase reaction at 1° C. until the sucrose substrate was 100% depleted (by about 70 hours).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method of producing trehalulose, the method comprising:
(a) contacting a sucrose solution with a sucrose isomerase enzyme to produce a sucrose-enzyme mixture; and
(b) incubating the sucrose-enzyme mixture of (a) at a temperature of less than 10° C. for a period of time sufficient to convert the sucrose in the sucrose-enzyme mixture to a product comprising trehalulose.

2. The method of claim 1, wherein the amount of sucrose in the sucrose-enzyme mixture is depleted by at least 75% as compared to the amount of sucrose in the sucrose solution of (a).

3. The method of claim 1, wherein the temperature of step (b) is from about 0.5° C. to less than 10° C.

4. The method of claim 1, wherein the temperature of step (b) is about 1° C.

5. The method of claim 1, wherein the sucrose solution of (a) comprises sucrose at an initial concentration of about 0.01M to a saturated solution.

6. The method of claim 1, wherein the enzyme is present in an amount from about 0.01 units to about 1000 units.

7. The method of claim 6, wherein the enzyme is present in an amount of about 600 units.

8. The method of claim 1, wherein the sucrose in the sucrose-enzyme mixture is reduced by an amount of about 75% to about 100%.

9. The method of claim 1, wherein the period of time sufficient to reduce the amount of sucrose in the sucrose-enzyme mixture is about 48 hours to about 96 hours.

10. The method of claim 1, wherein the period of time sufficient to reduce the amount of sucrose in the sucrose-enzyme mixture is about 70 hours.

11. The method of claim 1, wherein the method also produces isomaltulose and the ratio of trehalulose to isomaltulose is about 95:5.

12. The method of claim 1, wherein the sucrose isomerase enzyme is from Bemisia argentifoli, Erwinia rhapontici, Erwinia caratovera, Erwinia caratovera EcS1, Azotobacter vinelandii, Protoaminobacter rubrum, Pantoea dispersa, Serratia plymuthica, Klebsiella planticola, Klebsiella sp., Klebsiella terrigena, Pseudomonas mesoacidophila, Agrobacterium radiobacter, Pseudomonas mesoacidophila, Pseudomonas mesoacidophila (MX-45), Pseudomonas putida, Pimelobacter spp., Thermus sp. ATCC 43814, Thermus sp. ATCC 43815, Thermus aquaticus, Thermus filiformis, Thermus rubis, or any combination thereof.
